(19) United States
(12) Patent Application Publication
(10) Pub. No.: US 2006/0252152 A1
(43) Pub. Date: Nov. 9, 2006

(54) Attachment of cells to surfaces

(76) Inventors: James Van Alstine, Uppsala (SE); Hans Berg, Uppsala (SE); Asa Bjurling, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(21) Appl. No.: 10/571,193

(22) PCT Filed: Oct. 5, 2004
(86) PCT No.: PCT/SE04/01414

§ 371 Date: Mar. 8, 2006

(30) Foreign Application Priority Data
Oct. 6, 2003 (SE) 0302652-3

(51) Int. Cl. C12N 1/20; C12N 5/02; C12N 5/00; C12M 3/00

(52) U.S. Cl. 435/404; 435/253.6; 435/289.1

(57) ABSTRACT

The present invention relates to a microcarrier onto the surface of which a cationic compound has been immobilised via a guanidine group. The microcarrier is capable of attachment of cells, e.g. via charge-based interaction, and is advantageously used as a support in the culture of cells. Said compound may comprise one or two amino acids, such as arginine (Arg) or a dipeptide. The invention also relates to a method of preparing a polycationic microcarrier, which method comprises to immobilise a compound that comprises at least one guanidine group to an epoxide-activated substrate.

### TECHNICAL FIELD

The present invention relates to the field of microcarriers that are capable of attaching and maintaining cells during culture. More specifically, the invention relates to microcarriers that are comprised of a porous substrate that has been coated with a compound that enhances cell attachment. The invention also encompasses a method of preparing such microcarriers and a process of cell culture.

### BACKGROUND

The use of microcarrier supports to facilitate growth of biological cells has a long and varied history. Early such systems for effecting cell growth in useable quantities have included various dishes and flasks. Efforts intended to increase the quantity of cell production per unit apparatus volume by increasing culture surface area have resulted in the use of large trays. Some of these early cell-growth systems are still in use for applications where small-scale, high labour content, cell culture methods suffice, such as in hospitals and universities.

However, for large-scale processes, alternative methods are needed. One popular cell culture method is the roller bottle system. Essentially, a roller bottle is a cylindrical container arranged to contain a small amount of nutrient media. In operation, the roller bottle is rotated slowly about its longitudinal axis whereby the nutrient media is continually caused to wet the entire interior surface of the bottle, on which cell growth is achieved. A plurality of such roller bottles can be operated on a roller rack.

Another popular approach is microcarrier systems. Although other techniques have been introduced in recent years, such as membrane systems, the microcarrier systems still appear to be the most widely used cell culture method wherein anchorage dependent cell growth can be achieved at commercially advantageous production rates. Achieving large scale in this manner is superior to achieving it by replication, as is the case with roller bottles and other known systems. In addition, microcarrier bioreactor systems are well suited for automated large-scale cultivation of anchorage dependent cells.

Microcarrier development started in the late nineteen sixties when it was demonstrated that dextran beads could be used as a substrate for the growth of anchorage-dependent cells in a suspension culture mode. The first microcarriers were based on the use of common cationic, strong base, ion-exchange groups, such as diethylaminoethane (DEAE). Since then, a number of different materials including glass, polystyrene plastic, acrylamide, solid collagen, porous collagen, cellulose and liquid fluorocarbons have been successfully used as microcarriers.

Microcarriers with one or more adhesive peptides attached to the surface through covalent or noncovalent linkages have also been suggested. For example, U.S. Pat. No. 4,578,079 (La Jolla Research Foundation) discloses an illustrative composition which when immobilised on a substrate will promote attachment of cells. The composition disclosed is a small fragment of the protein fibronectin, and more specifically a polypeptide fragment that includes the sequence Arg-Gly-Asp attached to at least one other amino acid. Thus, the disclosed tetrapeptide composition has substantially the same cell attachment activity as fibronectin, which is based on biospecific affinity interaction. An obvious disadvantage with this composition is that it is bioactive, which means that its presence due for example to leeching into process streams may be undesired in certain applications such as in the pharmaceutical or diagnostic industry. The fragment claimed in U.S. Pat. No. 4,578,079 was according to the specification identified in an experiment wherein the design was to selectively synthesise peptides smaller than a previous fragment exhibiting cell attachment activity. It is stated in U.S. Pat. No. 4,578,079 that this approach permitted the determination of the smallest fragment showing activity. Furthermore, U.S. Pat. No. 6,180,610, which is a continuation of the above discussed '079, concludes that even though the tetrapeptide is the determinant, optimum size of the polypeptide is about a hexapeptide which includes the defined tetrapeptide.

It is also known that single amino acids can be used to provide groups that interact with desired target molecules, such as in chromatography. For example, the weak anion exchanger Arginine Sepharose™ (Amersham Biosciences, Uppsala, Sweden) is comprised of the amino acid Arg coupled to a particulate agarose matrix, predominantly through its α-amino group. Thus, Arginine Sepharose™ is provided commercially as a chromatography gel, wherein the average particle size in general is substantially smaller than the sizes required for microcarriers.

Other coatings that enhance the cell attachment properties of microcarriers have been presented. An illustrative example is Cytodex™1 (Amersham Biosciences, Uppsala, Sweden), which is comprised of a cross-linked dextran substrate coated with diethylaminoethane (DEAE) cellulose, which is a polycationic coating known to electrostatically bind cells. However, the cell attachment and growth properties are not optimal for all cell types, such as for cells with low plating-efficiency or differentiated or sensitive cell types. Consequently, there is still a need of improved alternative kind of microcarriers.

To further improve cell attachment properties, microcarriers coated with more complex compounds have been presented. An illustrative example is Cytodex™ 3 (Amersham Biosciences, Uppsala, Sweden), which like Cytodex™ 1 is comprised of a cross-linked dextran substrate, but is coated with a layer of acid-denatured porcine collagen. However, the mammalian origin of the collagen could limit use of Cytodex™ 3 in certain applications.

U.S. Pat. No. 6,214,618 relates to the field microcarrier beads for cell culture, and more specifically to the problem of avoiding animal proteins in such products. The suggested solution is a microcarrier bead made of a styrene copolymer with a trimethylamine exterior which has been washed in an acidic solution to make the beads compatible for cell culture.

U.S. Pat. No. 6,378,527 (Chondros Inc.) relates to a surgical procedure wherein chondrocytes are taken from a patient and rapidly multiplied and transplanted into said patient for cartilage repair. In order to multiply the chondrocytes, they are cultured, plated and finally grown onto a scaffold comprised of a polysaccharide derivative, which derivative is obtained by cross-linking a polysaccharide, such as dextran, with a polyamine. More specifically, the polysaccharides are first oxidised to the dialdehyde derivatives which is then reacted with non-toxic polyamines to form imine crosslinking, which can be further hydrogenated to the more stable amine bonds. An advantage of the disclosed microcarriers is that they allow proliferation of chondrocytes and formation of Collagen type II. Another advantage is that when utilised in vitro, the microcarriers will degrade to non-toxic components leaving chondrocytes in place to form cartilage tissue.

Obviously, there is still a need of alternative and/or improved microcarriers for cell culture, as well as of novel methods of preparing such microcarriers. There is also a more general need of improving the interaction between cells and other surfaces. This includes analytical surfaces used in a variety of applications such as drug screening, bioactive compound research, cell surface receptor studies, and monitoring of environmental contaminants.

### BRIEF DESCRIPTION OF THE PRESENT INVENTION

One aspect of the present invention is to provide a novel microcarrier, which allows cell attachment and cell culture. This can according to the present invention be achieved by a microcarrier onto the surface of which a cationic compound has been immobilised via a guanidine group, e.g. via charge-based interaction.

Another aspect of the invention is to provide a microcarrier for cell attachment and culture, which does not contain any mammalian-derived products.

A further aspect of the invention is to provide a microcarrier for cell attachment and culture, which is easy to control for toxicity and contaminants.

An additional aspect of the invention is to provide a method of preparing a novel polycationic microcarrier that allows cell attachment and cell culture. This can according to the present invention be achieved by a method, wherein a compound that comprises at least one guanidine group is contacted with an epoxide-activated substrate surface for immobilisation thereon.

Yet a further aspect of the invention is to provide process of cell culture, wherein the cells are cultured at the surfaces of one or more microcarriers coated with a cationic compound in an environment that provides for viability, said cells being attached to the microcarriers via guanidine groups provided by the cationic coating.

Further aspects and advantages of the invention will appear from the detailed description that follows.

### BRIEF DESCRIPTION OF THE DRAWINGS

FIG. 1 shows how the cell number changes with time in cultivation of Vero cells on a microcarrier according to the invention as explained in Example 1.

FIG. 2 shows how the cell number changes with time in cultivation of Vero cells on a microcarrier according to the invention as explained in Example 1.

### DEFINITIONS

The term “microcarrier” is used herein in its conventional sense for a particulate material used to support cell culture. Thus, in the present application, the term “microcarrier” will be used for a substrate to which a compound that enables cell attachment has been immobilised.

The term “polycationic” microcarrier means that the net charge of the microcarrier surface is positive.

The term “substrate” means the core of a carrier, i.e. a material to which cells can be attached.

The term “surface” of a substrate includes both the external surface of the substrate and, if porous, the pore surfaces.

### DETAILED DESCRIPTION OF THE INVENTION

A first aspect of the present invention relates to a microcarrier onto the surface of which a cationic compound has been immobilised via a guanidine group. In the present context, it is understood that the term “compound” means that an amount of said compound has been immobilised to the microcarrier surface rather than a single molecule thereof. Thus, since a plurality of molecules of said compound has been immobilised to each microcarrier, preferably as a coating, each microcarrier will present a positive net charge and can consequently be denoted “polycationic”.

In one embodiment, the present microcarrier is capable of attachment of cells via charge-based interaction between the cationic compound and the cells. In this context, it is understood that the term “attachment” means that the cells will be anchoraged sufficiently well to allow viability thereof. In one embodiment, the cationic compound interacts with proteins and preferably with tryptophan side chains. In another embodiment, the interactions are a mixture of the above-mentioned.

The present cationic compound, which has been immobilised onto the substrate surface by grafting to provide a coating, is essentially non-reactive, which means that in practise it will act to attach cells but not to any substantial level participate in reactions with other species. Further, the present compound is biocompatible in the sense that it will not have any deleterious effect on attached cells or other biological substances in the environment. Hence, the compound is non-toxic. In the present context, “attachment” of cells, also known as anchorage of cells, means that the cells are sufficiently strongly bound to be cultured.

As is well known, cells are generally not viable in high pH environments. Thus, in one embodiment, the microcarrier according to the present invention will present a surface wherein the pH is about 7. In a specific embodiment, the immobilised cationic compound forms a weakly basic coating on the substrate.

In the preferred embodiment of the present microcarrier, the immobilised compound comprises one or two amino acids. In an advantageous embodiment, the compound comprises an amino acid characterised by not being able to change charge and by not providing any self-catalysing formation of amide linkages with acid-bearing moieties. In a specific embodiment, the amino groups of the amino acid are reactive and capable of charge alteration via carbamate formation.

In one embodiment, the immobilised compound comprises any single amino acid except lysine (Lys). In order to provide the above-discussed weakly basic coating of the micro-carrier surface, the compound may comprise one or more basic amino acids. Thus, in an advantageous embodiment, the compound is arginine (Arg). Due to the acidic carboxylate group of Arg, this embodiment provides a microcarrier with a lower surface pH, and consequently a more favourable pH for cell culture, than the above discussed commercially available DEAE-coated microcarriers. A specific embodiment is a microcarrier to which has been immobilised an arginine-based compound, preferably an arginine to which a buffering acidic group has been attached. Such a compound is defined by the general formula Arg-X, wherein X is the buffering acidic group. In yet another embodiment, the compound is comprised of two or more different amino acids and consequently there will be two or more different ligands immobilised to the substrate surface. For example, the compound can be comprised of a mixture of arginine and aspartic acid.

In an alternative embodiment, the immobilised compound is, or comprises, a dipeptide. Thus, in one embodiment, the dipeptide is as arginine-glutamic acid (Arg-Glu) or arginine-aspartic acid (Arg-Asp). However, other dipeptides are also envisaged, as long as they provide the herein-disclosed properties necessary for cell attachment and growth to the microcarrier. In a specific embodiment, one or more further groups, such as buffering groups, can be attached to the dipeptide.

Thus, in summary, in one embodiment, the immobilised compound is selected from the group that consists of a single amino acid, such as Arg; a mixture of two amino acids, such as Arg and Asp; or a dipeptide, such as Arg-Glu, Arg-Asp or Arg-Arg. In the best embodiment, the immobilised compound is selected from the group that consists of a single amino acid, such as Arg; and a dipeptide, such as Arg-Glu, Arg-Asp or Arg-Arg.

The amino acid(s) and dipeptide(s) used according to the present invention are advantageously obtained from commercial sources, such as from Merck (arginine: Merck ref. 797; Arg-Glu: Merck ref. 798). Alternatively, they are easily prepared by the skilled person in this field using well-known recombinant or chemical techniques, such as solid phase synthesis. In brief, solid phase synthesis is commenced from the C-terminus of the peptide by coupling to a protected alpha-amino acid to a suitable resin; see e.g. U.S. Pat. No. 4,244,946.

Thus, the compound immobilised to the substrate of the present microcarrier is about the size of one amino acid, or possible two amino acids coupled to a dipeptide. In view of the conclusions of the above discussed U.S. Pat. No. 4,578,079 (La Jolla Research Foundation), which were that the smallest possible fragment of fibronectin should be a tetrapeptide or preferably even a hexapeptide, the finding of the present invention is clearly unexpected.

In addition, the attachment of cells to the present microcarrier is apparently based on charge-related interactions, and accordingly the compound is positively charged to enable attachment of negatively charged cells. This is a difference from the fibronectin fragments disclosed in the above discussed '079, such as Arg-Gly-Asp, which as stated therein has substantially the same biospecific affinity-based cell attachment activity as fibronectin.

In an alternative embodiment of the present microcarrier, the immobilised compound comprises a purine, or a mixture of purines. In a specific embodiment, the purine is adenine or guanine. Thus, the compound may comprise a nucleotide or nucleotide-related compound.

Since each guanidine group will act to help bind cells, the compound immobilised on the substrate surface can be considered as a ligand, the functional group of which is the guanidine group. In a specific embodiment of the microcarrier, the ligand concentration is in the range of 0.1-3.0 μmole/mg dry microcarrier. Thus, in illustrative embodiments, the ligand concentration is e.g. 0.2; 0.4; 0.7; 1.2; 2.5 or 3.0 μmole/mg dry microcarrier. In a specific embodiment wherein the immobilised compound is Arg, the ligand concentration is about 0.7 μmole/mg dry microcarrier. In an alternative embodiment, wherein the immobilised compound is a dipeptide, the ligand concentration is of approximately the same magnitude. An illustrative gravimetric density of the present microcarriers is in the range of 1.02-1.05 g/cm³, but lighter or heavier materials may be more suitable for specific applications.

In an advantageous embodiment, the substrate of the present microcarrier is a cross-linked carbohydrate, or comprised to an essential part of such a carbohydrate. In a specific embodiment, the microcarrier is made from a material selected from the group that consists of agarose, agar, cellulose, dextran, chitosan, konjac, carrageenan, gellan and alginate. Thus, in an advantageous embodiment, the present microcarrier is a gel. In the most advantageous embodiment, the microcarriers exhibit a porous structure that encourages cell growth into the bead, while the providing maximum nutrient availability.

The microcarrier materials according to the invention can easily be prepared according to standard methods, such as inverse suspension gelation (S Hjertén: Biochim Biophys Acta 79(2), 393-398 (1964). Alternatively, the base matrices are commercially available products, such as Sepharose™ FF (Amersham Biosciences, Uppsala, Sweden). In average, the microcarriers according to the invention present an average particle size of 40-300, such as 100-250 μm, for example about 230 μm (in 0.9% NaCl). In the most preferred embodiment, the present microcarriers are at least about 170 μm (in 0.9% NaCl).

In an alternative embodiment, the present microcarrier is comprised of cross-linked synthetic polymers, such as styrene or styrene derivatives, divinylbenzene, acrylamides, acrylate esters, methacrylate esters, vinyl esters, vinyl amides etc., which may themselves be modified to effect optimal immobilisation of the compound, i.e. ligand coupling, and cell culture.

In the most advantageous embodiment of the microcarrier according to the invention, the compound has been immobilised via a secondary amine to the substrate surface. Further details as regards the methods used for preparing a microcarrier according to the invention will be provided below in the context of the second aspect of the invention.

The present invention also encompasses the use of the above-described microcarrier(s) for cell culture. Thus, one aspect of the invention is a cell culture support comprised of at least one microcarrier according to any one of the preceding claims.

A second aspect of the present invention relates to a method of preparing a polycationic microcarrier by contacting a compound that comprises at least one guanidine group with an epoxide-activated substrate to provide immobilisation of the compound. The epoxide-activation can be basic, such as at pH 9, or acid catalysed. In this field, epoxide-activation of a substrate is a well known and commonly used immobilisation technique; see e.g. Immobilized Affinity Ligand Techniques, Hermanson et al, Greg T. Hermanson, A. Krishna Mallia and Paul K. Smith, Academic Press, INC, 1992. The immobilisation is most advantageously performed in a pH-controlled solution. Further details regarding the microcarrier prepared by use of the method according to the invention may be found above in relation to the first aspect of the invention.

The substrate can be any one of the above-discussed microcarrier materials, such as essentially spherical porous beads made from a cross-linked polysaccharide. In a step preceding the above-discussed epoxide-activation, a carbohydrate substrate is allylated, e.g. as described in the experimental part below. Contrary, if the substrate is made from a synthetic polymer, allyl groups will in general already be present and hence no allylation step will be required.

The compound immobilised is also as described in the context of the first aspect of the invention. Thus, in one embodiment, the immobilised compound is selected from the group that consists of a single amino acid, such as Arg; a mixture of two amino acids, such as Arg and Asp; or a dipeptide, such as Arg-Glu, Arg-Asp or Arg-Arg. In the best embodiment, the immobilised compound is selected from the group that consists of a single amino acid, such as Arg; and a dipeptide, such as Arg-Glu, Arg-Asp or Arg-Arg.

In the case where the compound is a dipeptide, the coupling can be a grafting reaction from a solution comprising both amino acids, i.e. a stepwise coupling of the first and then the second amino acid. Methods for immobilisation of compounds to the present kind of support surfaces are well known to the skilled person in this field.

A third aspect of the present invention relates to a method of attachment of cells to a surface, wherein a cationic compound comprising at least one guanidine group is used to attach cells to said surface. In one embodiment, the attachment is via charge-based interaction. In the most preferred embodiment, the compound is arginine (Arg). The surface can for example be the surface of a microcarrier, membrane, cloth, chip, such as a microchip, capillary, such as a microcapillary, or a vessel. In a specific embodiment, the present surface is the surface of a cloth that after attachment of cells can be used for various applications. The present method is e.g. useful for analytical purposes, in a production process, and/or for medical applications, as illustrated by tissue for treating burn victims.

In addition, the present invention relates to a method for localising cells for high throughput screening (HTS), which comprises a method as defined above in accordance to the third aspect of the invention.

A fourth aspect of the present invention relates to a process of cell culture, wherein the cells are cultured at the surfaces of one or more microcarriers coated with a cationic compound in an environment that provides for viability, said cells being attached to the microcarriers via guanidine groups provided by the cationic coating. In one embodiment, the attachment of cells is based on charge-based interaction. In the most advantageous embodiment, the microcarrier is as described above. In this context, the skilled person will understand that the term to provide for viability means that suitable temperature, nutrient feed and further parameters are maintained for each cell type. Thus, the microcarriers to which cells have been attached are maintained in solubilised or suspended form in a suitable vessel, preferably with temperature control and stirring. The cells that are cultured according to the invention may be any eukaryotic or prokaryotic cells, preferably eukaryotic cells, such as mammalian cells, e.g. recombinant cell lines, plant cells etc. In one embodiment, the cells are truly anchorage-dependent cells, such as primary cells or cells with epithelioid morphology. In an especially advantageous embodiment, the cells are cells with low plating-efficiency or differentiated or sensitive cell types such as hepatocytes or endocrine cells. In the experimental part below, this aspect of the invention is illustrated with a fibroblast cell line known as Vero cells, which is a strictly anchorage dependent fibroblast cell from African green monkey kidney.

In one embodiment, the present process comprises the further step of harvesting viable cells from said microcarriers. In one embodiment, in a subsequent step, the cultured cells are used for analytical and/or medical purposes. In an alternative embodiment, in a subsequent step, the cultured cells are used to support culture of virus, bacteria, molds, fungi or algae. This embodiment is advantageous in processes related to vaccine preparation and other applications.

Finally, the present invention relates to a microcarrier onto the surface of which gelatine originating from fish has been immobilised. Gelatine is a complex mixture of materials, but the proteins in it are expected to have large amounts of cationic peptides and some proteins. The substrate onto which the fish gelatine has been immobilised can be any of the ones discussed above in relation to the first aspect of the invention. Since there is no mammalian protein present on such a microcarrier, this aspect of the invention is advantageous in view of the available Cytodex™ 3 (Amersham Biosciences, Uppsala, Sweden) microcarriers. In addition, the gelatine may provide growth factors that enhance cell growth and may provide mechanical protection for attached cells when microcarriers collide with each other or with culture container surfaces.

### DETAILED DESCRIPTION OF THE DRAWINGS

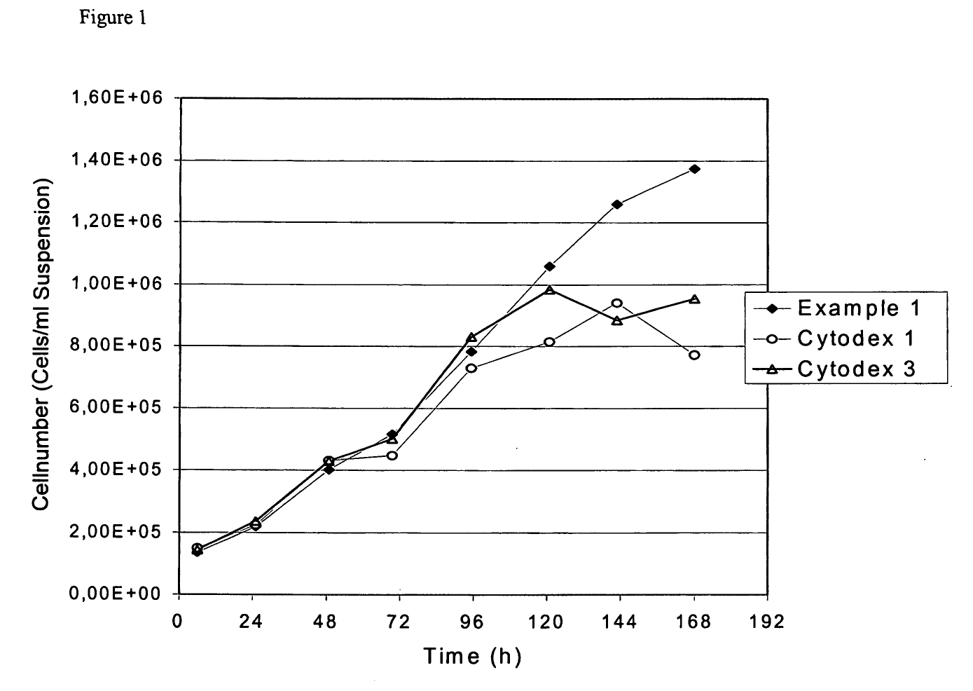

FIG. 1 shows how the cell number changes with time in cultivation of Vero cells on a microcarrier according to the invention as explained in Example 1. In FIG. 1, cell number (the number of cells/ml suspension) is shown on the Y-axis while time is shown on the X-axis. At approximately 168 hours, the uppermost line (diamonds), which illustrates cultivation using the present invention, has reached about 1.40E+06; while the comparative Cytodex™1 (circles), which is a commercial cross-linked dextran particle (Amersham Biosciences, Uppsala, Sweden), reaches about 8.00E+05 and the other comparative example, Cytodex™3 (triangles), a commercial cross-linked dextran particle with a gelatine coating (Amersham Biosciences, Uppsala, Sweden) reaches a value just below 1.00E+06 at the corresponding point of time. Thus, FIG. 1 clearly shows that the microcarrier according to the invention can provide an essentially higher cell number, and hence productivity, than the commercial microcarriers.

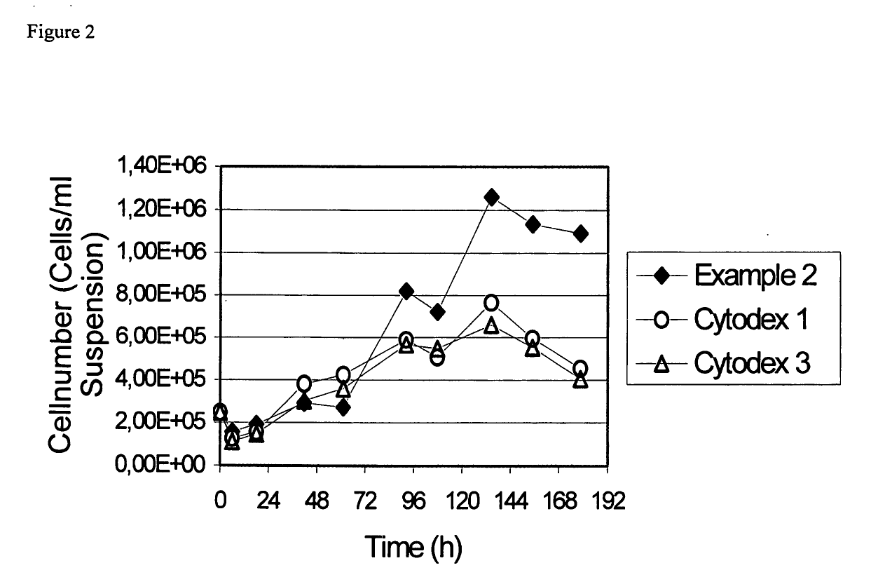

FIG. 2 shows how the cell number changes with time in cultivation of Vero cells on a microcarrier according to the invention as explained in Example 1. The diagram shown in FIG. 2 is analogue to the one of FIG. 1, except that the diamond signs denote the results obtained with a microcarrier according to example 2. Again, the microcarrier according to the invention shows a significant improvement in productivity as compared to the commercial products.

### EXPERIMENTAL PART

The present examples are provided for illustrative purposes only, and should not be construed as limiting the present invention as defined by the appended claims. All references given below and elsewhere in the present specification are hereby included herein by reference.

### EXAMPLE 1

Allylation of Sephadex™ G-50 Fine

32 g dry Sephadex™ G-50 Fine (Amersham Biosciences, Uppsala, Sweden) was mixed with 388 ml water in a glass container and left to swell for 1 h. The swelling of Sephadex ™ G-50 Fine are approximately 12 ml/g dry weight

The swollen gel was transferred to a three-necked round-bottom flask equipped with a mechanical stirrer. 44 g Na₂SO₄ was added to the round-bottom flask under stirring. The slurry was heated up to 30° C. and maintained for 1.5 h at 30° C. 80 ml NaOH (50% w/w) and 0.6 g NaBH₄ were added. The slurry was heated to 50° C. 80 ml allylglycidyl ether (AGE) was added. The reaction was continued over night at 50° C.

The reaction was stopped by neutralising to pH 6.5-7.5 by adding of acetic acid (60% w/w). The gel was washed on a glass filter with 4 gel volumes water, 3 gel volumes ethanol and finally with 6 gel volumes of water.

Allyl content was determined on a RADIOMETER ABU 93 TRIBURETTE with 0.1 M AgNO₃ according to standard methods. The allyl content was determined to 101 μmole/ml gel.

Arginine coupling of allylated Sephadex™ G50 Fine

250 ml of the allylated gel was transferred to a three-necked round-bottom flask equipped with a stirrer. 63 ml water and 10.3 g NaAc*3H₂O was added. Bromine water was added drop by drop until a persistent yellow colour was obtained. The reaction was continued for 5 min. The reaction was terminated by addition of sodium formate until the yellow colour had disappeared. The slurry was stirred for 30 min. The gel was then dry suck on a glass filter.

The gel was added to a round-bottom flask containing a solution of 251 ml water and 36.9 g L-Arginine. The slurry was heated to 45° C. and the pH was adjusted to pH 11.4 with NaOH (50% w/w). The pH was measured after 30 and 60 min and was adjusted to pH 11.4. The reaction was continued over night at 45° C.

The gel was washed on a glass filter with alternately one gel volume 0.1 M Tris, pH 8, and one gel volume 0.1 M NaOAc, pH 4, in totally eight cycles. Finally the gel was washed with 8 gel volumes water.

Chloride ion capacity was determined on a RADIOMETER ABU 93 TRIBURETTE with 0.1 M HCl according to standard methods. The chloride ion capacity was determined to 0.58 μmole/mg dry gel.

### EXAMPLE 2

Arginine Coupling of Allylated Sephadex™ G50 Fine

Analogous to Example 1, but using 50 ml of the allylated Sephadex™ G50 Fine from Example 1, 12.5 ml water, 2.06 g NaAc*3H₂O, 50 ml water and 7.33 g L-Arginine.

Chloride ion capacity was determined on a RADIOMETER ABU 93 TRIBURETTE with 0.1 M HCl according to standard methods. The chloride ion capacity was determined to 0.58 μmole/mg dry gel.

### EXAMPLE 3

### 3.1 Cultivation of Vero Cells in Different Cultivation Systems

All different microcarrier types (based on Cytodex™ carriers) were made in the same way, as described below. Under these conditions, a confluent layer means approx. 1×10⁵ cells/cm² and the critical cell number is 1-2×104⁴ cells/cm² for inoculation over a longer period of time. These facts are for T-flasks.

All operations were done under a hood under aseptic conditions with pyrogen free materials. The culture medium was always at 37° C. and at a pH of approx. 7.2.

3.1.1 Cultivation of Vero Cells in T Flasks

In case of a monolayer of Vero cells in a T flask (Nunclon™ Δ Surface; Nunc Brand Products), the cells were detached by trypsin or with the chelating agent ethylenediaminetetra-acetic acid (EDTA).

First, the supernatant was poured off in a waste bottle and the cells were washed once with PBS (wo Ca²⁺/Mg²⁺). In the next step, trypsin/EDTA was added to the T flask according table 1 below and distributed over the cell layer.

TABLE 1
Cultivation of Vero cells in T flasks

| | | Volume | |
|---|---|---|---|
| T-flask area | Volume medium | trypsin/EDTA | Volume inhibitor |
| [cm²] | [ml] | [ml] (see 3.3.3) | [ml] (see 3.3.4) |
| 25 | 10 | 0.3 | 0.06 |
| 80 | 30 | 1 | 0.2 |
| 175 | 50 | 2 | 0.4 |

After an incubation of several minutes at room temperature, the cells were detached by knocking to the T flask and immediately after the trypsin inhibitor was added according table 1. In the next step, the conditioned volume medium was added (see table 1). Then, the cells were divided under the terms of the split ratio. The Vero cells were passaged two times per week at a ratio of 1:3 (calculation of the required volume on base of culture surface).

The cells were cultivated in an incubator with 9% (v/v) CO₂ and a humidified atmosphere at 37° C.

3.1.2 Cultivation of Vero Cells in Roller Bottles

For inoculation of an 850 cm² roller bottle (Corning), a confluent culture in a 175 cm² T-flask was used. The cells were detached as described in 3.1.1. above. The cells were re-suspended in 200 ml conditioned medium and transferred to the roller bottle. The roller was aereted with 120 ml sterile CO₂ and cultivated at 37° C. on a roller apparatus at 0.2 rpm.

When cells from a roller culture are to be detached, the procedure would be similar. The supernatant was poured off in a waste bottle and the cell layer was washed once with PBS (wo CA²⁺/Mg²⁺). The PBS was also poured off into the waste bottle and the volume trypsin/EDTA was added according table 2.

TABLE 2
Cultivation of Vero cells in roller bottles

| | | Volume | |
|---|---|---|---|
| Roller bottle | Volume medium | trypsin/EDTA | Volume inhibitor |
| [cm²] | [ml] | [ml] (see 3.3.3) | [ml] (see 3.3.4) |
| 850 | 200 | 10 | 2 |

After an incubation time of several minutes at room temperature, the cells were knocked from the roller and the inhibitor was added according table 2. The cells were resuspended in the conditioned medium (see table 2). Then, the cell suspension can be divided to several rollers and filled up with medium to 200 ml and to each roller 120 ml sterile CO₂ was added. The cells were cultivated at 37° C. at a rotation speed of 0.2 rpm.

3.1.3 Detachment of Vero Cells with EDTA

For inoculation of a microcarrier culture, the cells should be detached from the surface by the EDTA method.

A 0.02% (w/w) EDTA solution was diluted from the 0.16% (w/w) stock solution in sterile PBS (wo Ca²⁺/Mg²⁺) and warmed up to 37° C. in a water bath. The supernatant of the confluent cell layer was poured off and the cells were washed once with PBS (wo Ca²⁺/Mg²⁺). The cells were detached as described in 3.1.1 and 3.1.2 according table 3.

TABLE 3
Detachment of Vero cells with EDTA

| | EDTA | | | |
|---|---|---|---|---|
| Culture ves- | 0.02% | Temperature | | |
| sel | [ml] | [° C.] | Time [min] | Note |
| T-flask | 4 | 37 | approx. 10 | — |
| 175 cm2 | | | | |
| Roller | 20 | 37 | approx. 10 | 0.2 rpm |
| 850 cm2 | | | | |

It was not necessary to add any kind of inhibitor, because the EDTA will be complexed by the bivalent ions from the medium. The same amount of medium was added as described in table 1 or table 3.

Then the cell suspension can be divided to several vessels and be cultivated as described in 3.1.1 and 3.1.2.

### 3.2. Cultivation of Vero Cells on Microcarrier in Spinner Flasks

All operations were performed in a hood under aseptic and pyrogen free conditions.

3.2.1 Equilibration and Dosage of Microcarriers for Spinner Cultures

At least the volume from eq. 1 of microcarriers was provided into a 50 ml tube. After the carriers had sedimented, the PBS was removed and replaced by the same amount of medium. The carriers were resuspended and after sedimentation, the medium was also removed. This procedure was repeated four times. Finally, the dilution factor for the PBS should be higher than 10.

$${V}_{Carrier} = \frac{n \times {V}_{Culture} \times c \times SV}{1000} \qquad Eq . 1$$

V_{Carrier} . . . Volume of swollen carrier [ml]
V_{Culture} . . . Final culture volume [ml]
n . . . Carrier concentration [g/l]
SV . . . Swelling Volume of the microcarrier [ml/g]

After the last washing step and carrier sedimentation, the medium was pulled off by a pipette, so that 50% of the final volume was the sedimented microcarriers. From this suspension, the volume according eq. 2 was transferred into a spinner.

$${V}_{Spinner} = \frac{2 \times c \times {V}_{Culture} \times SV}{1000} \qquad Eq . 2$$

V_{Spinner} . . . Carrier suspension per spinner [ml]
V_{Culture} . . . Final culture volume [ml]
c . . . Carrier concentration [g/l]
SV . . . Swelling Volume of the microcarrier [ml/g]

The spinner with medium and carrier was equilibrated in an incubator over night (37° C.; 9% (v/v) CO₂).

3.2.2 Inoculation of Spinner Cultures

The cells were cultivated either in 175 cm² T flasks or in 850 cm² roller bottles as described in 3.1.1 and 3.1.2. The cells were detached from the rollers by the EDTA method (see 3.1.3). The cells were pooled from the different culture flasks and kept on 37° C. during the inoculation procedure. The number of flasks for an experiment was calculated according eq. 3.

$${N}_{Flasks} = \frac{{CC}_{Spinner} \times {V}_{Culture} \times n}{{A}_{Flask} \times {10}^{5}} \qquad Eq . 3$$

N_{Flask} . . . Number of flasks
CC_{Spinner} . . . Final cell concentration for inoculation
   [Cells/ml]
V_{Culture} . . . Final culture volume [ml]
N . . . Number of Spinners
A_{Flask} . . . Culture surface [cm²]
   175 cm² for T175 or 180 cm² for R850

The total cell concentration and the viability were estimated by the Trypan blue stain method.

The volume of the inoculum per spinner was calculated by eq. 4.

$${V}_{Inoc} = \frac{{V}_{Culture} \times {CC}_{Inoc} \times 100}{{CC}_{Total} \times {F}_{Viab}} \qquad Eq . 4$$

V_{Inoc} . . . Volume inoculum per spinner [ml]
V_{Culture} . . . Final culture volume [ml]
CC_{Total} . . . Cell concentration total inoculum
   [Cells/ml]
F_{Viab} . . . Viablility [%]

A final culture volume of 40 ml was chosen for all experiments, that means a headspace of approximately 200 ml gas atmosphere per spinner (125 ml flask; Techne).

For inoculation, the spinner was moved circular carefully and the clapper was fixed with a magnetic stirring bar. The inoculum was added to the carrier suspension drop by drop within 20 sec. After this procedure, the culture was mixed with an intermitted stirring profile 25 min 0 rpm and 5 min 35 rpm for 6 h at 37° C. on a spinner platform (Cellspin; Integra Biosciences). After this period of time, the culture was filled up to 40 ml with fresh medium and the continuous stirring at 35 rpm was started. At this point, the first sample was taken (see 3.2.3). Sampling was done every 24 h.

After 48 h, 20 ml of the medium was changed and each spinner was aerated with a gas mixture (75% N₂, 20% O₂, 5% CO₂; gas exchange 750 ml per spinner; Linde). From this moment on, every 24 h medium exchange and aeration was performed.

3.2.3 Sampling of Spinner Cultures

3.2.3.1 Estimation of Cell Concentration

For routine sampling, cell concentration by counting the released nuclei was used. In this method, cells growing on the microcarriers were incubated in a hypotonic solution and nuclei released by lysis were stained by crystal violet in this solution (Microcarrier cell culture; Pharmacia Biotech; 2000).

A homogenous sample of 1.5 ml was taken from the spinner culture. 1 ml of this sample was provided to a 1.5 ml Eppendorf tube and the carrier was sedimented by gravity; 0.5 ml of the total sample volume (1.5 ml) were for pictures (see 3.2.3.2). After that, the supernatant was sucked off by a pipette. According the expected cell concentration and 0.5 ml or 1.0 ml of the crystal violet solution (see 3.3.1) was given to the tube and mixed by vortexing the sample. This sample was incubated 1 h at 37° C. with vortexing from time to time.

After the incubation was finished, the released nuclei are counted by a heamocytometer and the cell concentration was calculated (Eq. 5).

These samples were stable for one week at 4° C.

$${CC}_{Spinner} = \frac{RN \times {V}_{CV} \times 10000}{SQ \times {V}_{Sample}} \qquad Eq . 5$$

CC_{Spinner} Cell concentration [Cells/ml Susp.]
RN Total number of counted nuclei
V_{CV} Volume crystal violet solution [ml]
SQ Number of cell counting squares
V_{Sample} Sample Volume [ml]
   3.2.3.2 Making Pictures of Microcarrier Cultures

Sampling was done as described in 3.2.3.1 and 0.5 ml of the sample was provided to a 1.5 ml Eppendorf tube. After sedimentation of the carriers by gravity, the supernatant was pulled off and 0.25 ml of the haematoxylin solution (see 3.3.2) was given to the carriers and mixed carefully. The tubes were incubated at room temperature for several hours (at least 2 h, better o/n). For microscopy, 301 μl of each sample was provided to one well of a 96 well plate (Cat. No.: 269620; Nunc Brand Products) and a picture was made by a digital camera at a microscope.

### 3.3. Solutions

3.3.1 Crystal Violet Solution for Counting Released Nuclei

| | | | | |
|---|---|---|---|---|
| Citric acid | 0.1 mol/l | 19.21 | g/l | FW_{C6H8O7} = 192.1 g/mol |
| Crystal violet | 0.1% | 1 | g/l | |
| RO- water | | | | |

The solution was filtrated through a filter paper and stored at 4° C.

3.3.2 Staining of Microcarriers with Heamatoxylin

| | | | | | |
|---|---|---|---|---|---|
| Heamatoxyling | 0.1% | (w/w) | 1 | g/l | |
| NaIO₃ | 0.02% | (w/w) | 0.02 | g/l | |
| KAl(SO₄)₂ × 12H₂O | 1% | (w/w) | 10 | g/l | FW = 474.38 g/mol |

Dissolved in 900 ml RO-water at room temperature and stirring o/n at room temperature.

The following substances were given to the solution after stirring.

| | | | | | |
|---|---|---|---|---|---|
| Chloralhydrate | 5% | (w/w) | 50 | g/l | FW_{C2H3Cl3O2} = 165.4 g/mol |
| Citric acid | 0.1% | (w/w) | 1 | g/l | FW_{C6H8O7} = 192.1 g/mol |

The solution was filled up to 1000 ml with RO-water and filtered through a paper filter.

The solution was stored at 6° C. and should be used within 2 years.

3.3.3 Trypsin Solution for Cell Detachment

| | | | | | |
|---|---|---|---|---|---|
| Trypsin | 0.1% | (w/w) | 1 | g/l | Gibco 27250-042 |
| EDTA | 0.02% | (w/w) | 0.2 | g/l | |

Dissolved in PBS (w/o Ca²⁺/Mg²⁺) and sterilised by filtration through a 0.2 μm filter. The solution was either stored at 4° C. or −20° C.

3.3.4 Trypsin inhibitor for cell detachment

| | | |
|---|---|---|
| Trypsin inhibitor | 1 mg/ml | Sigma T 6522 |

Dissolved in PBS (w/o Ca²⁺/Mg²⁺) and sterilised by filtration through a 0.2 μm filter. The solution was either stored at 4° C. or −20° C.

1. A microcarrier onto the surface of which a cationic compound has been immobilised via a guanidine group.

2. The microcarrier of claim 1, which is capable of attachment of cells via charge-based interaction between the cationic compound and the cells.

3. The microcarrier of claim 1, wherein the cationic compound provides a polycationic coating at the microcarrier surface.

4. The microcarrier of claim 1, wherein the cationic compound provides a weakly basic coating at the microcarrier surface.

5. The microcarrier of claim 1, wherein the cationic compound comprises one or two amino acids.

6. The microcarrier of claim 5, wherein the cationic compound consists of arginine (Arg).

7. The microcarrier of claim 5, wherein the cationic compound consists of a dipeptide.

8. The microcarrier of claim 7, wherein the dipeptide is arginine-glutamic acid (Arg-Glu) or arginine-aspartic acid (Arg-Asp).

9. The microcarrier of claim 1, wherein the cationic compound has been immobilised via a secondary amine to the microcarrier surface.

10. The microcarrier of claim 1, wherein the microcarrier is comprised of a cross-linked carbohydrate.

11. A cell culture support comprised of at least one microcarrier of claim 1.

12. A method of preparing a microcarrier, comprising contacting a compound that comprises at least one guanidine group with an epoxide-activated substrate surface and immobilizing the compound on the surface via the guanidine group.

13. The method of claim 12, wherein the compound comprises one or two amino acids.

14. The method of claim 13, wherein the compound consists of arginine (Arg).

15. The method of claim 13, wherein the compound consists of a dipeptide.

16. The method of claim 12, wherein the compound comprises one or more nucleotides.

17. The method of claim 12, wherein the substrate is a cross-linked carbohydrate.

18. (canceled)

19. A method of attachment of cells to a surface, comprising using a cationic compound including at least one guanidine group is used to attach cells to said surface.

20. The method of claim 19, wherein the attachment is via charge-based interaction.

21. The method of claim 19, wherein the cationic compound consists of arginine (Arg).

22. The method of claim 19, wherein the surface is the surface of a microcarrier, membrane, cloth, slide, chip, capillary or vessel.

23. (canceled)

24. A method for localizing cells for high throughput screening (HTS), which utilizes the method of claim 19.

25. A process of cell culture, comprising:
(a) attaching the cells to the surfaces of one or more microcarriers coated with a cationic compound; and
(b) culturing said cells in an environment that provides for viability, wherein said cells are attached to the surfaces of one or more microcarriers via guanidine groups provided by the cationic coating.

26. The process of claim 25, wherein the attachment of cells is based on charge-based interaction.

27. The process of claim 25, wherein the cationic compound consists of arginine (Arg).

28. The process of claim 26, further comprising a step of harvesting viable cells from said microcarriers.

29. The process of claim 26 further comprising a step of using the cells for analytical and/or medical purposes.

30. The process of claim 25, further comprising a step of using the cells to support culture of virus, bacteria, molds, fungi or algae.
